(19) United States
(12) Patent Application Publication
(10) Pub. No.: US 2015/0066126 A1
(43) Pub. Date: Mar. 5, 2015

(54) Fenestration Electrode to Treat Patients with Meniere's Disease

(71) Applicant: MED-EL Elektromedizinische Geraete GmbH, Innsbruck (AT)

(72) Inventors: Andreas Marx, Innsbruck (AT); Andreas Jäger, Reith bei Seefeld (AT); Helge Rask-Andersen, Uppsala (SE)

(21) Appl. No.: 14/468,518

(22) Filed: Aug. 26, 2014

Related U.S. Application Data
(60) Provisional application No. 61/871,357, filed on Aug. 29, 2013.

(51) Int. Cl. A61N 1/05

(52) U.S. Cl. CPC A61N 1/0526
USPC 607/137

(57) ABSTRACT

An implantable fenestration electrode delivers electrical stimulation signals for treatment of Meniere's disease. An electrode lead contains one or more signal wires for carrying a stimulation signal. An electrode tip at a terminal end of the electrode lead is configured for placement within a fenestration opening in an outer surface of a bony labyrinth of a patient with Meniere's disease without penetrating or impairing intra-labyrinthine membranes or neural tissue, and is adapted to deliver the stimulation signal via intra-labyrinthine fluid to the intra-labyrinthine neural tissue.

This application claims priority from U.S. Provisional Patent Application 61/871,357, filed Aug. 29, 2013, which is incorporated herein by reference.

### TECHNICAL FIELD

The present invention relates to implant systems for treatment of Meniere's disease, and specifically a stimulation electrode for such implant systems.

### BACKGROUND ART

The balance sensing functionality of the brain is developed based on neural signals from the vestibular structures of the inner ear, one on each lateral side of the body. As shown in FIG. 1, each inner ear vestibular labyrinth 100 has five sensing organs: the ampullae 108 of the three semi-circular canals—the horizontal (lateral) canal 103, the posterior canal 104, and the superior (anterior) canal 105—which sense rotational movement, and the utricle 106 and the saccule 107 in the vestibule 102, which sense linear movement.

FIG. 2 shows anatomical detail within a vestibular canal ampulla 108 which is connected at one end to the canal 206 and at the other end to the vestibule 205, and which contains endolymph fluid. The vestibular nerve endings 204 connect to the crista hair cells 203, the cilia ends 202 of which are embedded in the gelatinous cupula 201. When the head moves, the endolymph fluid within the ampulla 108 deflects the cupula 201, generating a sensing signal in the vestibular nerve endings 204 that is interpreted by the brain as the sense of balance.

When the head is stationary, the vestibular system generates neural activity at a certain rate that is transmitted by the vestibular nerve to the brain. When the head moves in a given direction, the vestibular system changes the neural activity rate on the affected nerve branch of the vestibular nerve which correlates with the head movement. Unfortunately some people suffer from damaged or impaired vestibular systems or from various diseases that affect intact vestibular systems such as Meniere's disease. Dysfunction of the vestibular system can cause problems such as unsteadiness, vertigo (feeling of rotation) and unsteady vision.

In the special case of Meniere's disease, the vestibular system is not structurally damaged or impaired, but rather it provides changing nerve activity rates that do not correlate with head movements. This results in severe dizziness, balance problems, loss of orientation and other patient issues. Some known treatments for severe cases of Meniere's disease result in permanent loss of vestibular function. For example, one conventional treatment of Meniere's disease is the use of ototoxic drugs to destroy vestibular hair cells. This treatment however results in a permanent loss of vestibular function although patients could benefit from a functional vestibular system between phases of Meniere's attacks and this treatment also endangers pre-existing hearing.

Another approach to treatment of Meniere's disease is to use electrical stimulation of vestibular neural tissue with a Meniere treatment implant. But there are some major differences between a conventional vestibular prosthesis and a Meniere treatment implant. A conventional vestibular prosthesis is intended to restore natural function of the vestibular system when the natural vestibular sensory function has been lost. This requires measuring head movements and providing corresponding stimulation patterns to the respective branches of the vestibular nerve. A Meniere treatment implant has no need to sense head movement but instead treats the Meniere's symptoms; for example, by blocking irregular vestibular nerve activity rate unrelated to head movements by providing a constant stimulation pattern to the vestibular nerve to reduce symptoms like dizziness in the patient during a Meniere's attack. So a conventional vestibular prosthesis and a Meniere implant have some major different requirements in their body interface.

Most conventional vestibular implant arrangements are based on use of an intra-labyrinthine electrode configured to be inserted into the vestibular labyrinth. But such an approach either will damage or at least risks damage to the delicate intra-labyrinth neural structures.

There also have been some proposals for use of vestibular stimulation electrodes that are placed outside the vestibular labyrinth. Such extra-labyrinthine stimulation electrodes directly approach in close proximity to individual ampullary nerve branches of the vestibular nerve. See, e.g., Wall et al., Eye Movements in Response to Electric Stimulation of the Human Posterior Ampullary Nerve, Ann Otol. Rhinol Laryngol. 116, 369-374, (2007); Feigl et al., Superior Vestibular Neurectomy: A Novel Transmeatal Approach for a Denervation of the Superior and Lateral Semicircular Canals, Otol. Neurotol. 30,586-591 (2009); Guyot et al., Eye Movements in Response to Electrical Stimulation of the Lateral and Superior Ampullary Nerves, Ann. Otol. Rhinol. Laryngol. 120,81-87 (2011); Guyot et al., Adaptation to Steady-State Electrical Stimulation of the Vestibular System in Humans Ann Otol. Rhinol Laryngol. 120,143-149 (2011); all incorporated herein by reference.

Such extra-labyrinthine electrodes have advantages and disadvantages compared to intra-labyrinthine electrodes. Some advantages are the preservation of the delicate intra-labyrinthine structures (reducing the risk of generating a sensorineural hearing loss) and the shorter distance to the targeted nerve branches. The disadvantages of extra-labyrinthine electrodes are related to the surgical accessibility of the ampullary nerve branches:
Drilling in close proximity to nerves increases the risk of damaging the nerve.
Approaching the lateral and superior ampullary nerve branches may require removal of parts of the ossicular chain, which results in a conductive hearing loss.
Since the lateral and superior ampullary nerve branches are in close proximity to the facial nerve, there is an increased risk of damaging the facial nerve and/or unintentionally stimulating it.

### SUMMARY

Embodiments of the present invention are directed to an implantable fenestration electrode that delivers electrical stimulation signals for treatment of Meniere's disease. An electrode lead contains one or more signal wires for carrying a stimulation signal. An electrode tip at a terminal end of the electrode lead is configured for placement within a fenestration opening in an outer surface of a bony labyrinth of a patient with Meniere's disease without penetrating or impairing intra-labyrinthine neural tissue, and is adapted to deliver the stimulation signal via intra-labyrinthine fluid to the intra-labyrinthine neural tissue (e.g., by perilymph and/or endolymph fluid).

An anchor mesh is located around the outer surface of the electrode lead near the electrode tip to engage the outer surface of the bony labyrinth to fixedly secure the electrode tip within the fenestration opening. The anchor mesh may lie parallel or perpendicular to the electrode lead and may be fixed (e.g. by screwing, gluing and/or by ingress into tissue) to the outer surface of the bony labyrinth. The electrode tip may have a spherical section outer surface and may be adapted for monopolar, bipolar, multipolar and/or parallel stimulation operation. And embodiments also include a Meniere treatment implant system having at least one fenestration electrode according to any of the above.

### BRIEF DESCRIPTION OF THE DRAWINGS

FIG. 1 shows the vestibular labyrinth of the inner ear.

FIG. 2 shows anatomical detail of a vestibular canal ampulla.

FIG. 3 A-B shows top plan and side cross-section views of an implantable fenestration electrode according to an embodiment of the present invention.

FIG. 4 shows a side cross-section view of an implanted fenestration electrode according to an embodiment of the present invention.

FIG. 5 shows a combined electrode including a cochlear implant electrode and three fenestration electrodes according to an embodiment of the present invention.

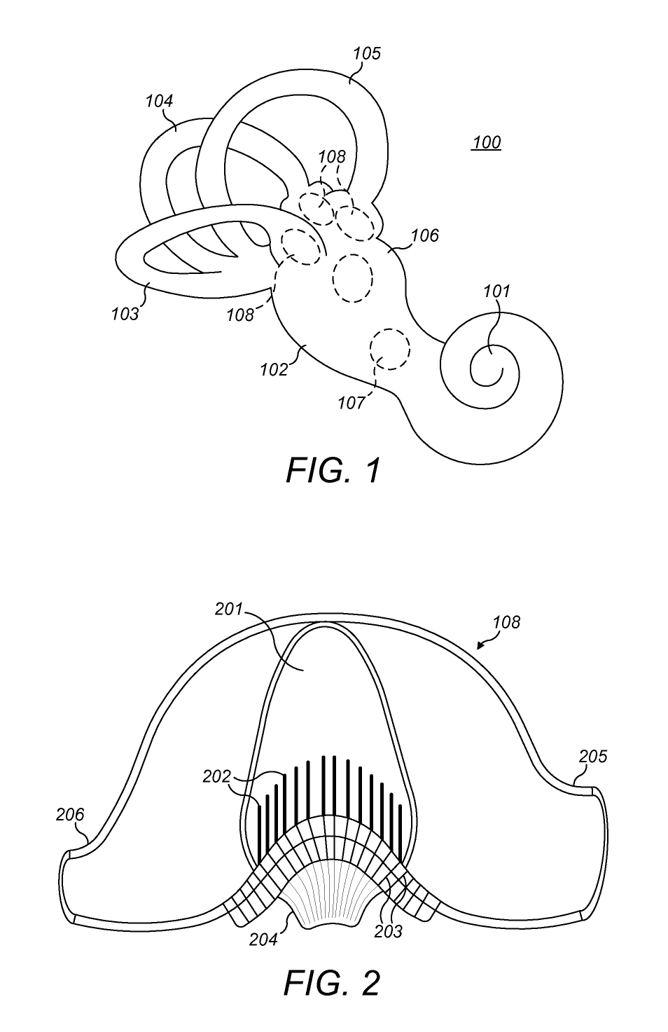

### DETAILED DESCRIPTION

Embodiments of the present invention are directed to a fenestration electrode that exploits the advantages from both intra- and extra-labyrinthine electrodes while avoiding their disadvantages. A fenestration is a drilled opening into the vestibular labyrinth performed during a labyrinthotomy surgery. The labyrinthotomy may either penetrate the endosteal layer that covers the bone on the inside of the vestibular labyrinth, or it may leave the endosteum intact. A stimulation electrode is placed within the fenestration to deliver stimulation signals for treatment of Meniere's disease.

The fenestration electrode is tailored to the specific requirements for stimulation electrode to treat Meniere's disease but might also be used for other vestibular disorders where treatment by means of providing electrical stimulation to the vestibular nerve is applied. The surgical technique to make a fenestration opening into the vestibular labyrinth is already known by surgeons from existing surgical techniques such as SCC plugging. The labyrinthotomy itself can be done in a way that preserves the function of the delicate anatomical structures within the labyrinth. The fenestration electrode uses the intra-labyrinthine fluids (endolymph and perilymph) as electrical conductors to deliver the stimulation signal to the inner ampulla. This allows the stimulation contact surface on the tip of the fenestration electrode to be placed at a safe distance from the delicate neural tissue, which reduces the risk of traumatic injury during implantation.

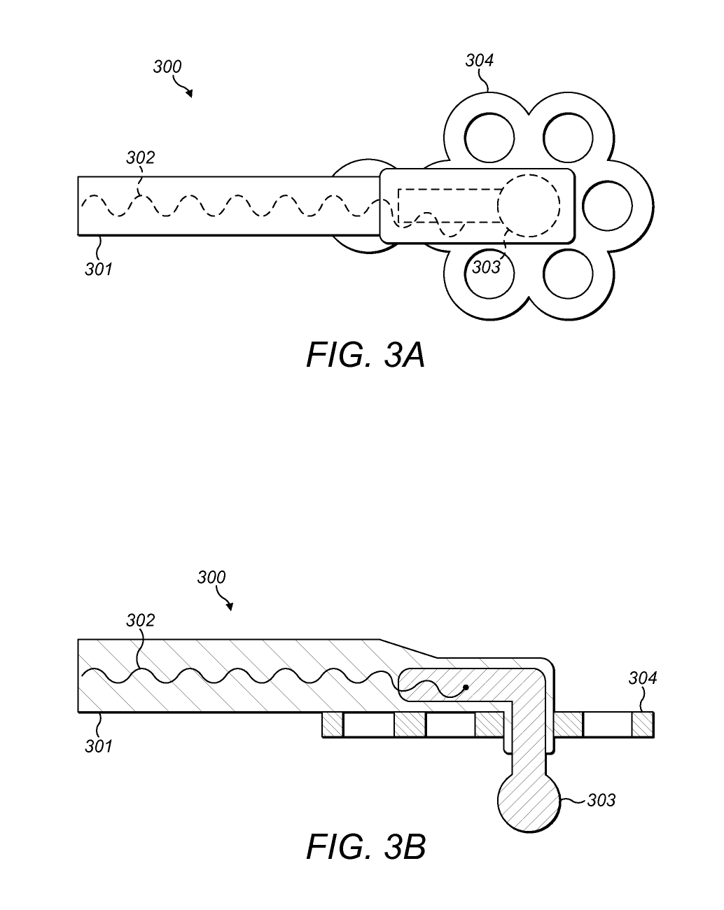

FIG. 3 A-B shows top plan and side cross-section views of an implantable fenestration electrode 300 according to an embodiment of the present invention. The fenestration electrode 300 includes an electrode lead 301 made of resilient and electrically insulating material and containing one or more signal wires 302 for carrying a stimulation signal. An electrode tip 303 is located at the terminal end of the electrode lead 301 and is configured for placement within the fenestration opening without penetrating or impairing the delicate intra-labyrinthine tissue (membranous labyrinth). In the embodiment shown in FIG. 3 A-B, the electrode tip 303 has a spherical section shape outer surface that makes electrical contact with either the endosteum or the intra-labyrinthine fluid, depending on the specific nature of the fenestration opening. In specific Meniere treatment implant systems, the electrode tip 303 may be adapted for monopolar, bipolar, multipolar and/or parallel stimulation operation.

An anchor mesh 304 is located around the outer surface of the electrode lead 301 near the electrode tip 303 to engage the outer surface of the bony labyrinth to fixedly and accurately secure the electrode tip 303 within the fenestration opening. For example the anchor mesh 304 may be glued to the outer surface of the bony labyrinth to seal the labyrinthotomy. The anchor mesh 304 may lie parallel or perpendicular to the electrode lead 301 depending on the specific geometry associated with the vestibular labyrinth canal location.

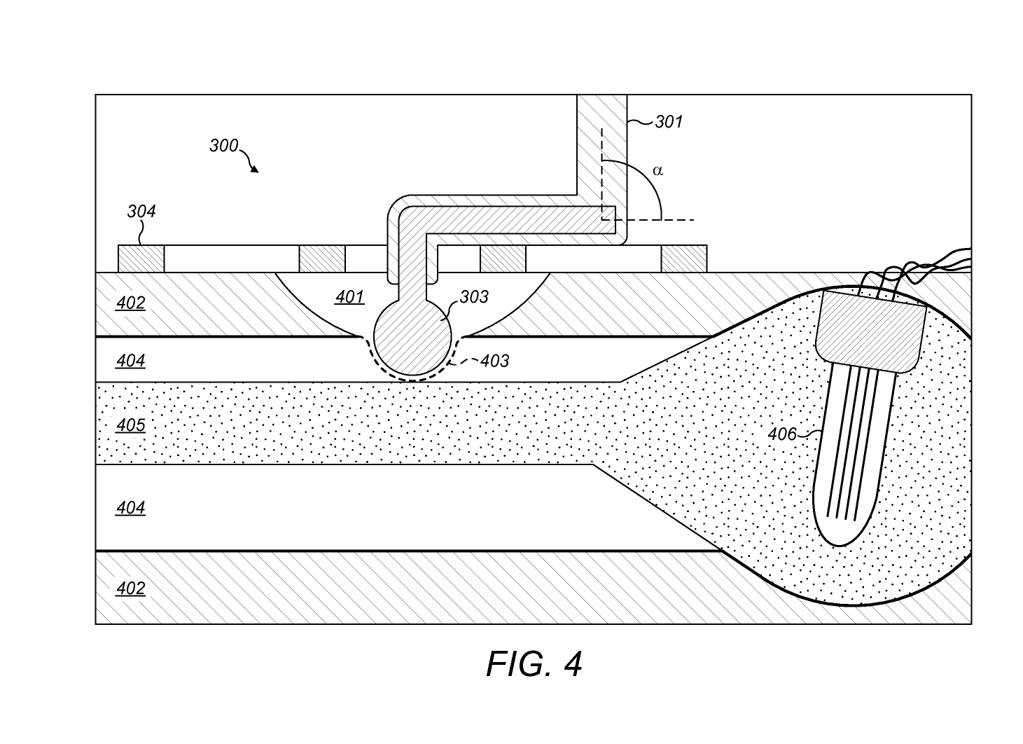

FIG. 4 shows a side cross-section view of an implanted fenestration electrode 300 according to an embodiment of the present invention. In the embodiment shown, the endosteum 403 remains intact within the fenestration opening 401 in the bone 402 of the vestibular labyrinth. During a Meniere's episode, the spherical end of the electrode tip 303 delivers the stimulation signal from an implanted stimulation module across the endosteum 403 to the perilymph 404 and endolymph 405 fluids within the vestibular labyrinth, which conduct and deliver the stimulation signal to the neural tissue of the ampulla 406 for vestibular sensation that treats the Meniere's symptoms.

The mesh 304 (about 0.2 mm thick) may have an upper and a lower mesh surface, the lower surface being the one facing the electrode tip 303. The distance of a tip lead section as defined from the lower mesh surface to where the electrode lead enters/is attached to the tip electrode 303, may be between 0.7 mm and 0.2 mm, more preferably between 0.5 mm and 0.3 mm, e.g., 0.4 mm. The length of the tip lead section is chosen such that the entire or a substantial portion of the electrode tip 303 may enter the space of the perilymph fluid 404 if such deep insertion is desired by the surgeon. In this case a portion of the electrode tip 303 may be moved laterally after insertion through fenestration opening 401 such that an edge of bone 402 at the fenestration opening 401 is between this portion of the electrode tip 303 and the mesh 304. For this purpose the electrode tip 303 may have a larger dimension than the tip lead section; e.g., if the electrode tip 303 has a spherical outer surface, its diameter may be greater than the cross section of the (cylindrical) tip lead section, e.g. 0.5 mm diameter of electrode tip 303 and 0.2 mm cross section of the tip lead section. Such parameters would provide additional securement (i.e. in addition to the mesh 304 fixed to bone 402) of the electrode tip 303 after implantation to prevent migration out of fenestration opening 401 over time. Insertion of the entire or a substantial portion of the electrode tip 303 into the space of perilymph 404 probably would cause rupture of endosteum 403. This little trauma may be outweighed, however, by secure functioning and save placement of electrode tip 303.

The electrode tip 303 extends from the end of the electrode lead 301 at a specific angle α between 0° and 90° that depends on the intended implantation site. This angle α eases surgical placement of the electrode tip 303 within the fenestration opening 401. For example, for placement in the superior canal, the electrode tip 303 may extend from the distal end of the electrode lead 301 as shown in FIG. 4 to be perpendicular to the plane of the anchor mesh 304. For placement in the lateral canal or the posterior canal, the electrode tip 303 may extend from the distal end of the electrode lead 301 in the plane of the anchor mesh 304.

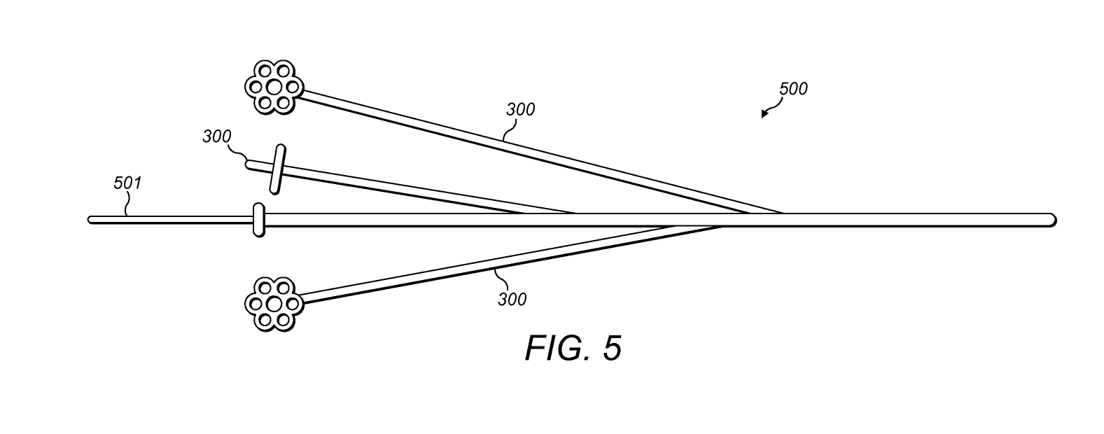

FIG. 5 shows a combined electrode 500 including a cochlear implant electrode 501 and three fenestration electrodes 300 according to an embodiment of the present invention. Each branch of such a combined electrode 500 may reflect a unique identifier characteristic such as color, specific shape of the anchor mesh, lead structure, etc. Each fenestration electrode 300 may be operated independently in monopolar configuration with a distant reference electrode (e.g., on skull bone like with a cochlear implant electrode). Or two or more fenestration electrodes 300 may be operated together in parallel (with a distant reference electrode), or in bipolar mode (without a distant reference electrode), or in some other multipolar mode.

The electrode disclosed in this application may also be beneficial for other treatments than that for Meniere disease, e.g. for the treatment of bilateral vestibular dysfunction, single sided vestibular dysfunction, vestibular migraine, or related diseases of the vestibular organs. In addition, it might also be beneficial in vestibular implants including sensors to record the attitude of the carrier's body or head as disclosed e.g. in U.S. Pat. No. 6,546,291 or WO 2011/088130, both documents incorporated herein by reference.

Although various exemplary embodiments of the invention have been disclosed, it should be apparent to those skilled in the art that various changes and modifications can be made which will achieve some of the advantages of the invention without departing from the true scope of the invention. This also includes use for other vestibular disorders than Meniere's disease where treatment by means of providing electrical stimulation to the vestibular nerve can be applied.

What is claimed is:

1. An implantable fenestration electrode for delivering electrical stimulation signals for treatment of Meniere's disease, the fenestration electrode comprising:
an electrode lead containing one or more signal wires for carrying a stimulation signal for treatment of Meniere's disease symptoms; and
an electrode tip at a terminal end of the electrode lead configured for placement within a fenestration opening in an outer surface of a bony labyrinth of a patient with Meniere's disease without penetrating or impairing intra-labyrinthine neural tissue, and adapted to deliver the stimulation signal via intra-labyrinthine fluid to the intra-labyrinthine neural tissue.

2. The fenestration electrode according to claim 1, further comprising:
an anchor mesh around an outer surface of the electrode lead near the electrode tip adapted to engage the outer surface of the bony labyrinth to fixedly secure the electrode tip within the fenestration opening.

3. The fenestration electrode according to claim 2, wherein the anchor mesh lies parallel to the electrode lead.

4. The fenestration electrode according to claim 2, wherein the anchor mesh lies perpendicular to the electrode lead.

5. The fenestration electrode according to claim 2, wherein the anchor mesh is adapted to be glued to the outer surface of the bony labyrinth.

6. The fenestration electrode according to claim 1, wherein the electrode tip has a spherical section outer surface.

7. The fenestration electrode according to claim 1, wherein the fenestration electrode is adapted for monopolar operation.

8. The fenestration electrode according to claim 1, wherein the fenestration electrode is adapted for bipolar operation.

9. The fenestration electrode according to claim 1, wherein the fenestration electrode is adapted for multipolar operation.

10. The fenestration electrode according to claim 1, wherein the fenestration electrode is adapted for parallel electrode operation.

11. The fenestration electrode according to claim 1, wherein the electrode tip is configured for delivery of the stimulation signal via perilymph fluid to the intra-labyrinthine neural tissue.

12. The fenestration electrode according to claim 1, wherein the electrode tip is configured for delivery of the stimulation signal via endolymph fluid to the intra-labyrinthine neural tissue.

13. A Meniere treatment implant system having at least one fenestration electrode according to any of claims 1-12.
